(19) United States
(12) Patent Application Publication
(10) Pub. No.: US 2024/0076606 A1
(43) Pub. Date: Mar. 7, 2024

(54) NOVEL MICROALGAE WITH HIGH PRODUCTIVITY OF ZEAXANTHIN AND LUTEIN

(71) Applicant: ASK LABS, Daejeon (KR)

(72) Inventors: Hee Sik Kim, Daejeon (KR); Yong Jae Lee, Daejeon (KR); Su Bin Park, Daejeon (KR)

(73) Assignee: ASK LABS, Daejeon (KR)

(21) Appl. No.: 18/273,584

(22) PCT Filed: Jan. 18, 2022
(86) PCT No.: PCT/KR2022/000879

§ 371 Date: Jul. 21, 2023

(30) Foreign Application Priority Data
Jan. 22, 2021 (KR) 10-2021-0009601

(51) Int. Cl. C12N 1/12; A61K 36/05; C12P 23/00

(52) U.S. Cl. CPC C12N 1/125; A61K 36/05; C12P 23/00; C12R 2001/89

(57) ABSTRACT

The present invention relates to HS-LZ microalgae of the genus Chlorella (Chlorella sp. HS-LZ), which are novel microalgae characterized by being able to produce zeaxanthin and lutein, and uses thereof.

### TECHNICAL FIELD

The present invention relates to microalgae of the genus Chlorella characterized by excellent growth rate and high productivity of zeaxanthin and lutein.

### BACKGROUND ART

Zeaxanthin is a type of carotenoid alcohol commonly found in nature. It plays an important role in the xanthophyll cycle and is mainly synthesized in plants. Zeaxanthin is one of the carotenoid components contained in the retina of the eye and is found in the center of the macular. It is known to play a crucial role in eye health. Zeaxanthin is one of the pigments with high economic value, as it is known to have an antioxidant function to protect the eyeball and to improve eye diseases such as cataract and macular degeneration. Lutein, like zeaxanthin, is one of the pigments widely known to be helpful for eye health. Lutein presents in large amounts in pigments such as plant chloroplasts and is also concentrated in the macula or lens of the eye. Lutein is known to help eye health by maintaining macular pigment density that can be reduced due to aging.

Due to the efficacy and utilization of the zeaxanthin and lutein, they possess high added value. Conventionally, a method of extracting zeaxanthin and lutein from marigold petals has been used for industrially produce them. However, marigold, as a flower crop, has disadvantages in that it has a long growth period and productivity is highly likely to be lowered due to the influence of climate or environmental disasters while growing in open cultivated land. Moreover, marigolds have issues in that they are cultivated in various parts of the world have various varieties for each cultivation area, and the amount of pigment obtained from petals varies greatly depending on the regional climate, cultivation conditions, harvesting methods and extraction methods. Moreover, the content of pigments, such as lutein, specified in raw materials is different from 2% to 100%, causing extreme deviations (Miroslav Sivel et al., “Lutein Content in Marigold Flower (Tagetes erecta L.) Concentrates used for Production of Food Supplements”, Czech J. Food Sci., Vol. 32, 2014, No. 6:521-525 (2014)). Therefore, from the perspective of the industrial production of zeaxanthin and lutein, it is difficult to secure a steady supply of raw materials and the quality of powder or pellets as raw materials is not uniform.

Accordingly, studies on the production methods of zeaxanthin and lutein using microalgae have been conducted, which allow easier incubation due to less influence by the environment, relatively uniform quality of pigments produced, and year-around stable production of pigments. However, the pigments produced by microalgae either do not contain zeaxanthin or contain too many different types of pigments other than zeaxanthin or lutein, so that additional separation and purification of zeaxanthin and lutein was inevitable. Therefore, there is a growing need to develop novel microalgae capable of producing zeaxanthin or lutein with higher efficiency.

### DETAILED DESCRIPTION

### Technical Objective

An objective of the present invention is to provide novel microalgae capable of producing large amounts of zeaxanthin and lutein pigments, which were conventionally extracted and produced from plants such as marigolds, and their use for producing zeaxanthin and lutein.

In addition, an objective of the present invention is to provide a method for producing zeaxanthin or lutein using the novel microalgae as described above.

### Means for Solving the Problems

In order to achieve the above objectives, one aspect of the present invention provides HS-LZ microalgae of the genus Chlorella (Chlorella sp. HS-LZ) deposited under the accession number KCTC 14308BP.

Another aspect of the present invention provides a method for producing zeaxanthin or lutein, comprising the steps of culturing the HS-LZ microalgae of the genus Chlorella (Chlorella sp. HS-LZ) deposited under the accession number KCTC 14308BP; and obtaining pigments from the culture medium of the microalgae.

Another aspect of the present invention provides a composition for producing zeaxanthin or lutein comprising the HS-LZ microalgae of the genus Chlorella (Chlorella sp. HS-LZ) deposited under the accession number KCTC 14308BP.

Another aspect of the present invention provides a pharmaceutical composition for preventing or treating eye diseases, comprising the HS-LZ microalgae of the genus Chlorella (Chlorella sp. HS-LZ) deposited under the accession number KCTC 14308BP or an extract thereof.

Another aspect of the present invention provides a health functional food for preventing or improving eye disease, comprising the HS-LZ microalgae of the genus Chlorella (Chlorella sp. HS-LZ) deposited under the accession number KCTC 14308BP or an extract thereof.

### WORKING EFFECT OF THE INVENTION

A novel HS-LZ microalgae of the genus Chlorella of the present invention, unlike general chlorella, can grow in seawater, freshwater and brackish water and exhibit a high growth rate therein. Therefore, it is characterized by high biomass productivity and excellent production of pigments and fats. Unlike conventional HS2 microalgae of the genus Chlorella, the HS-LZ microalgae are characterized in that it can produce zeaxanthin. The pigments produced by the HS-LZ microalgae primarily consist of zeaxanthin and lutein and hardly contain other pigments including chlorophyll a, by which the process for separating and purifying zeaxanthin and lutein can be greatly simplified.

Compared to the method of extracting pigments from marigold petals, which is a conventional method used to produce zeaxanthin or lutein, the method for producing zeaxanthin or lutein using microalgae of the present invention omits the harvesting process requiring additional labor. In addition, due to the unicellular nature of the microalgae of the present invention, the resulting products become homogeneous which provides consistent production of the pigments.

Furthermore, the HS-LZ microalgae of the present invention remarkably increase the productivity of zeaxanthin and lutein under certain conditions, allowing consistent production of the pigments with high efficiency.

The effects of the present invention are not limited to those mentioned above, and other effects not mentioned herein will be clearly understood by those skilled in the art from the following descriptions.

### BRIEF DESCRIPTION OF DRAWINGS

FIG. 1 shows the treatment of EMS on HS2 microalgae of the genus Chlorella to induce mutations and the subsequent smear on a solid medium, confirming the color changes of the medium for each microalgae colony.

FIG. 2 shows the result of culturing of the HS2 microalgae mutant colony of the genus Chlorella, in which the solid medium is treated with EMS and the color is changed to a color other than green, and then the colony is cultured in liquid medium under the conditions without light (dark) and the conditions with light (light).

FIG. 3 shows the HPLC analysis results of pigment components produced from wild-type HS2 microalgae of the genus Chlorella (Wild), from variants M2 to M7, and from the HS-LZ of the genus Chlorella of the present invention.

FIG. 4 shows the graphs comparing the contents of each component (lutein, zeaxanthin, etc.) in the pigments produced from wild-type HS2 microalgae of the genus Chlorella (Wild), from variants M2 to M7, from the HS-LZ of the genus Chlorella of the present invention, and from the HS-LZ cultured under stress conditions in which no organic carbon source exists (LZ-st).

### BEST MODE FOR CARRYING OUT THE INVENTION

Hereinafter, the present invention will be described in detail.

### 1. Novel Microalgae of the Genus Chlorella

The present invention provides novel microalgae of the genus Chlorella.

The microalgae of the present invention are the HS-LZ microalgae of the genus Chlorella (Chlorella sp. HS-LZ) deposited under the accession number KCTC 14308BP.

The HS-LZ microalgae of the genus Chlorella are novel microalgae improved from HS2 microalgae of the genus Chlorella (the accession number KCTC 13108BP). The HS2 microalgae of the genus Chlorella exhibit a high growth rate in environments with various carbon sources or in environments of freshwater, seawater, and brackish water, and demonstrate excellent characteristics in terms of biomass, pigment, and lipid productivity. The HS-LZ microalgae of the genus Chlorella of the present invention can also exhibit the advantages and characteristics of the HS2 microalgae of the genus Chlorella.

The HS-LZ microalgae of the genus Chlorella may comprise a polynucleotide comprising the nucleotide sequence of SEQ ID NO:1. Specifically, the 18s rDNA of the HS-LZ microalgae of the genus Chlorella may comprise the nucleotide sequence of SEQ ID NO:1. The nucleotide sequence of SEQ ID NO:1 is the same as the 18s rDNA nucleotide sequence of HS2 microalgae of the genus Chlorella. Accordingly, the HS-LZ microalgae of the genus Chlorella of the present invention may be identified into genus Chlorella.

The HS-LZ microalgae of the genus Chlorella can produce zeaxanthin. Unlike conventional microalgae of the genus Chlorella, such as HS2 microalgae of the genus Chlorella which cannot produce zeaxanthin, the HS-LZ microalgae of the genus Chlorella of the present invention can produce zeaxanthin.

The HS-LZ microalgae of the genus Chlorella can produce lutein. The HS-LZ microalgae of the genus Chlorella of the present invention may have higher productivity of lutein compared to conventional microalgae of the genus Chlorella, such as HS2 microalgae of the genus Chlorella.

The zeaxanthin and lutein are pigments of high economic value in that they are known to retain activities helpful to eye health. Since the microalgae of the present invention are characterized in that they can produce the pigments with high purity, they may be useful for the production of zeaxanthin or lutein. Furthermore, the microalgae of the present invention may be used for preventing or treating eye diseases or for improving eye health.

The HS-LZ microalgae of the genus chlorella may produce zeaxanthin or lutein and accumulate them within cells or may release them outside of cells.

The pigments produced by the HS-LZ microalgae of the genus Chlorella may contain at least one of zeaxanthin and lutein in an amount of 90 parts by weight or more based on 100 parts by weight of the pigment. Specifically, the pigments may contain zeaxanthin and/or lutein in an amount of 92 parts by weight or more, 95 parts by weight or more, 96 parts by weight or more, 97 parts by weight or more, 98 parts by weight or more, 99 parts by weight or more, or 99.5 parts by weight or more based on 100 parts by weight of the pigment. The pigments may not contain other pigments other than zeaxanthin and lutein. The pigments produced by the HS-LZ microalgae of the genus Chlorella may not contain at least one selected from the group consisting of chlorophyll a, chlorophyll b, neoxanthin and violaxanthin. Conventional Chlorella microalgae, for example HS2 microalgae of the genus Chlorella, may not produce zeaxanthin. Even if they produce zeaxanthin, they may also produce other pigments (the chlorophyll a, chlorophyll b, neoxanthin, violaxanthin, etc.) in addition to zeaxanthin and lutein in large quantities. Thus, the pigments other than zeaxanthin and lutein may be included in an amount of more than 10 parts by weight among the total pigments. In contrast, the pigments produced by the HS-LZ microalgae of the genus Chlorella of the present invention may contain at least one of zeaxanthin and lutein in an amount within the above ranges, and zeaxanthin and/or lutein account for the majority of the pigments. As such, the advantages of the present invention reside in that the process of separation and purification to obtain zeaxanthin or lutein may be simplified or omitted.

The HS-LZ microalgae of the genus Chlorella may produce 1 mg/g to 5 mg/g of zeaxanthin per dry weight of the microalgae culture medium. Specifically, the HS-LZ microalgae of the genus Chlorella may produce 1.2 mg/g to 4.5 mg/g, 1.5 mg/g to 4 mg/g, 1.9 mg/g to 3.6 mg/g, 1.8 mg/g to 2.2 mg/g, 1.9 mg/g to 2 mg/g, 3 mg/g to 4 mg/g, 3.3 mg/g to 3.8 mg/g or 3.5 mg/g to 3.7 mg/g of zeaxanthin.

The HS-LZ microalgae of the genus Chlorella may produce 0.2 mg/g to 3 mg/g of lutein per dry weight of the microalgae culture medium. Specifically, the HS-LZ microalgae of the genus Chlorella may produce 0.5 mg/g to 2.5 mg/g, 0.6 mg/g to 2 mg/g, 0.7 mg/g to 1.5 mg/g, 0.5 mg/g to 1 mg/g, 0.6 mg/g to 0.9 mg/g, 0.7 mg/g to 0.9 mg/g, 1.2 mg/g to 1.5 mg/g, or 1.3 mg/g to 1.4 mg/g of lutein.

### 2. A Method for Producing zeaxanthin or lutein

The present invention provides a method for producing zeaxanthin or lutein.

The method for producing the zeaxanthin or lutein according to the present invention comprises the steps of culturing the HS-LZ microalgae of the genus Chlorella (Chlorella sp. HS-LZ) deposited under the accession number KCTC 14308BP; and obtaining pigments from the culture medium of the microalgae.

The HS-LZ microalgae of the genus Chlorella are the same as the one provided in the section “1. Novel microalgae of the genus Chlorella.”

The HS-LZ microalgae of the genus Chlorella of the present invention may be characterized in that they can produce zeaxanthin or lutein in high purity. Therefore, a high percentage of zeaxanthin or lutein may be produced when going through the steps of culturing the HS-LZ microalgae of the genus Chlorella and extracting the pigments therefrom. The pigment components produced by the HS-LZ microalgae of the genus Chlorella are mostly zeaxanthin and lutein, and other pigments such as chlorophyll a, chlorophyll b, neoxanthin, and violaxanthin are hardly contained. As such, the advantages of the present invention reside in that zeaxanthin or lutein may be produced in high purity.

The step of culturing the HS-LZ microalgae of the genus Chlorella may be culturing under light or non-light conditions. The HS-LZ microalgae of the genus Chlorella of the present invention may produce zeaxanthin or lutein regardless of the presence or absence of light. Thus, unlike other conventional chlorella microalgae, which are generally cultured under conditions given light, the advantages of the present invention reside in that zeaxanthin or lutein may be produced even under dark conditions where no light is given.

The step of culturing the HS-LZ microalgae of the genus Chlorella may be culturing in a conventional culture medium of Chlorella. For example, it may be culturing in a medium containing sugars, amino acids, organic acids, and the like. Specifically, the medium may contain glucose, dextrose, sucrose, fructose, maltose, lactose, molasses, glutamic acid, malic acid, citric acid, and the like. The HS-LZ microalgae of the genus Chlorella may be cultured in a medium further containing a nitrogen source and an inorganic salt. The nitrogen source may be nitrate, nitrite, urea, ammonium chloride, ammonia, peptone, tryptone, yeast extract, beef extract, malt extract, potato extract, or NPK fertilizer, and the inorganic salt may be Na⁺, K⁺, mg²⁺ or Ca²⁺ but is not limited thereto. The medium may be a complex fertilizer for plants, a complex medium for microbial fermentation, a BG11 medium, and the like that are commonly used. The water contained in the medium for culturing the HS-LZ microalgae of the genus Chlorella may be seawater, brackish water or fresh water. For example, it may be fresh water but is not limited thereto. The type of water included in the medium may be appropriately selected in consideration of a process for producing zeaxanthin or lutein, culturing conditions of the HS-LZ microalgae of the genus Chlorella, and the like.

In addition, the step of culturing the HS-LZ microalgae of the genus Chlorella may comprise culturing the microalgae for 3 to 14 days under conditions in the absence of an organic carbon source. For example, culturing may be conducted for 3 to 10 days, 4 to 9 days, or 5 to 8 days. When microalgae are cultured under stress conditions without an organic carbon source, the production amount of pigments of the microalgae may be increased and the production amount of zeaxanthin and lutein may be increased. The step of culturing the HS-LZ microalgae of the genus Chlorella may significantly increase the production amount of zeaxanthin and lutein by culturing in conditions with an organic carbon source to increase the number of cells, and then by culturing in a medium without an organic carbon source.

The step of obtaining the pigments may comprise the step of disrupting the microalgal cells in the culture medium. As the method for disrupting the microalgal cells, any method may be used to disrupt the microalgal cells as long as the cells are disrupted without destroying or losing activity of the pigments within the cells, specifically, zeaxanthin or lutein. For example, cells may be disrupted by sonication.

The step of obtaining the pigments may comprise the step of extracting the pigments from the microalgae. For the extraction of the pigments, any extraction method known to those skilled in the art may be used. As the extraction method, all known conventional extraction methods such as solvent extraction (including low-temperature dark conditions extraction), ultrasonic extraction, filtration and reflux extraction may be used. Specifically, the pigments may be prepared by using solvent extraction or reflux extraction. The extraction process may be repeated several times, and the subsequent steps such as concentration or lyophilization may be additionally performed. In particular, the obtained extract may be concentrated under reduced pressure to obtain a concentrate, and then a high-concentration extract powder may be prepared using a grinder after lyophilizing the concentrate. The extraction solvents may include hexane, benzene, toluene, diethyl ether, chloroform, ethyl acetate, methylene chloride, 1,4-dioxane, tetrahydrofuran, acetone, acetonitrile, dimethylformamide, dimethyl sulfoxide, acetic acid, alcohols having 1 to 4 carbon atoms including methanol, ethanol, propylene glycol, propyl alcohol, butylene glycol, butyl alcohol, glycerin, and the like, may be used but are not limited thereto.

The method for producing zeaxanthin or lutein of the present invention may not comprise the step of removing chlorophyll a, chlorophyll b, neoxanthin, and violaxanthin. That is, it may not comprise the step of separating only zeaxanthin and lutein separately. Since the pigments produced by the HS-LZ microalgae of the genus Chlorella may not contain or may contain very small amount of pigments other than zeaxanthin and lutein, zeaxanthin and lutein may be obtained in high purity without a process of removing the pigments such as chlorophyll a, chlorophyll b, neoxanthin, violaxanthin, etc. However, in order to obtain higher purity of zeaxanthin and lutein from the pigments, the additional step of separating zeaxanthin and lutein may be performed as necessary. In this case, high-performance liquid chromatography (HPLC) may be used but is not limited thereto. In addition, if only one of zeaxanthin and lutein is wanted to be produced as needed, the step of separating zeaxanthin or lutein may be further performed.

### 3. Uses of the HS-LZ Microalgae of the Genus Chlorella

The present invention provides a use for the production of zeaxanthin or lutein of the HS-LZ microalgae of the genus Chlorella (Chlorella sp. HS-LZ).

A composition for producing zeaxanthin or lutein of the present invention comprises the HS-LZ microalgae of the genus Chlorella (Chlorella sp. HS-LZ) deposited under the accession number KCTC 14308BP.

The HS-LZ microalgae of the genus Chlorella is the same as the one provided in the section “1. Novel microalgae of the genus Chlorella”. The HS-LZ microalgae of the genus Chlorella are microalgae capable of producing zeaxanthin or lutein, and hardly produce other pigments other than zeaxanthin and lutein among the pigments produced. Therefore, separation and purification processes of zeaxanthin or lutein may be simplified or omitted, so that high-purity zeaxanthin or lutein may be easily produced. In addition, unlike lutein produced by marigold, lutein produced from the HS-LZ microalgae of the genus Chlorella does not have fatty acids linked to both ends of the lutein molecule, so that it does not involve a process for removing fatty acids. Considering the characteristics of the HS-LZ microalgae of the genus Chlorella, a composition containing the microalgae may be usefully used to produce zeaxanthin or lutein.

In addition, the present invention provides a use of the HS-LZ microalgae of the genus Chlorella (Chlorella sp. HS-LZ) or an extract thereof for preventing, treating or improving eye diseases.

A pharmaceutical composition for preventing or treating eye diseases and a health functional food for preventing or improving eye diseases of the present invention comprise the HS-LZ microalgae of the genus Chlorella (Chlorella sp. HS-LZ) deposited under the accession number KCTC 14308BP or an extract thereof.

As described above, since the HS-LZ microalgae of the genus Chlorella or the extract thereof contain zeaxanthin or lutein, the composition may be used for various uses according to the physiological activity of zeaxanthin or lutein. Specifically, the composition may be used for preventing, treating and improving for eye diseases.

The eye diseases may be at least one selected from consist of decreased vision (e.g., aged-related decreased vision), vision loss, cataracts, glaucoma, peroxidation of the eye, retinal damage, dry eye syndrome, diabetic retinopathy, macular degeneration including non-exudative age-related macular degeneration and exudative age-related macular degeneration, choroidal neovascularization, retinopathy, acute and chronic macular neuroretinopathy, central serous chorioretinopathy, macular edema, acute multiple placoid pigment epitheliopathy, birdshot retinopathy, posterior scleritis, tortuous choroiditis, subretinal fibrosis, uveitis syndrome, retinal arterial occlusion, central retinal vein occlusion, disseminated intravascular coagulation, branched retinal vein occlusion, hypertensive fundus change, ocular ischemic syndrome, retinal artery microvascularization, Coates disease, idiopathic macular telangiectasis, hemiretinal vein occlusion, papillary phlebitis, central retinal artery occlusion, branch retinal artery occlusion, frosted branch angiitis, sickle cell retinopathy, vascular pattern retinopathy, familial exudative vitreoretinopathy, Eales disease, proliferative vitreoretinopathy, proliferative diabetic retinopathy, tumor-related retinal disease, congenital thickening of the retinal pigment epithelium (RPE), posterior uveal melanoma, choroidal hemangioma, choroidal osteoma, choroidal metastasis, combined hamartoma of the retina and retinal pigment epithelium, retinoblastoma, fundus angioproliferative tumor, retinal astrocytoma, intraocular lymphoid tumor, myopic retinal degeneration, acute retinal pigment epithelitis, and glaucoma, but is not limited thereto. Any eye-related disease or disorder known to be prevented, treated, or improved by zeaxanthin or lutein may be applicable.

The term “preventing” refers reducing the risk of contracting a disease or disorder. It means all actions that suppress or delay the onset of a disease by inhibiting the progression of one or more clinical symptoms of the disease in a subject who is susceptible to expose or contract to the disease but does not yet suffer from the disease or does not show symptoms of the disease.

The term “treating” refers alleviating of a disease or disorder. It means all actions that ameliorate or beneficially alter the symptoms of a disease by arresting or reducing the progression of the disease or one or more clinical symptoms thereof.

The term “improving” refers all actions that ameliorate or beneficially alter the diseases or the symptoms of disorder.

The pharmaceutical composition of the present invention may be administered orally or parenterally to mammals, comprising livestock and humans, for example it may be applied intravenous and arterial, intramuscular, subcutaneous, intraperitoneal, mucosal or topical (e.g., eye drops), ocular, transdermal, and the like.

The pharmaceutical composition may be formulated into oral administration formulations, for example tablets, troches, lozenges, aqueous or oily suspensions, prepared powders or granules, emulsions, hard and soft capsules, syrups or elixirs, and the like. Binders such as lactose, saccharose, sorbitol, mannitol, starch, amylopectin, cellulose or gelatin; excipients such as dicalcium phosphate; disintegrants such as corn starch or sweet potato starch; and lubricating oil such as magnesium stearate, calcium stearate, sodium stearyl fumarate or polyethylene glycol wax may be contained for formulating the pharmaceutical composition into dosage forms such as tablets and capsules. In the case of a capsule formulation, a liquid carrier such as fatty oil may be contained in addition to the above-mentioned materials.

When the pharmaceutical composition of the present invention is administered parenterally, it is applied as subcutaneous injection, intravenous injection, intramuscular injection and intrathoracic injection method, and is applied to mucous membranes or topically. In such cases, it is administered by liquid dosage forms suitable for parenteral administration such as dispersions, suppositories, powders, aerosols (nasal sprays or inhalants), eye drops, gels, suspensions (aqueous or non-aqueous liquid suspensions, oil-in-water or water-in-oil emulsions), solutions, and the like. In order to form a formulation for parenteral administration, the composition may be mixed in water with a stabilizer or a buffer to form a solution, which may be formulated as a unit administration type of ampoule or vial.

The effective dosage of the HS-LZ microalgae of the genus Chlorella or extract thereof of the present invention may be diversified by factors such as formulation method, administration method, patient's age, weight, sex, medical condition, food, administration time, route of administration, excretion rate and reaction sensitivity. Generally, the effective dosage ranges from 1 to 20 mg/day per 1 kg of body weight of an adult patient, or specifically, 5 to 10 mg/day. It may be administered multiple times a day at regular time intervals based on the judgement of the doctor or pharmacist, or specifically, 2 to 3 divided doses per day.

The health functional food of the present invention may adopt the HS-LZ microalgae of the genus Chlorella or extract thereof in a range of 0.01 to 50% (w/w) based on the total weight, or specifically, 1 to 30% (w/w). Various foods may be produced by commonly known methods by adding the HS-LZ microalgae of the genus Chlorella or extract thereof to common favorite foods, that is, noodles such as ramen and fresh noodles, tofu, cereals, breads, chewing gum, candy, confectionery, and the like. It may be added as an edible food additive. In addition, it may be formulated into general formats such as tablets, granules, pills, hard capsules, soft capsules, or liquid formulations. Furthermore, it may be produced into fresh juice, pouches, beverages, or teas. In addition to the above components, other components may be appropriately selected and formulated by a person skilled in the art depending on the formulation.

### MODE FOR CARRYING OUT THE INVENTION

Hereinafter, the present invention will be described in detail in view of embodiments. While the following embodiments illustrate the present invention specifically, the scope of the present invention is not limited by the following embodiments.

### Embodiment 1 Preparation of the HS-LZ Microalgae of the Genus Chlorella (Chlorella sp. HS-LZ)

The HS-LZ microalgae of the genus Chlorella (Chlorella sp. HS-LZ) of the present invention were prepared by artificially inducing mutations in HS2 microalgae of the genus Chlorella (Chlorella sp. HS2). Through this, the microalgae which characterized in excellent productivity of zeaxanthin and lutein could be prepared.

The HS2 microalgae of the genus Chlorella, which is used as the parent strain for improvement, can grow in both freshwater, seawater and brackish water environments and exhibit excellent growth rates. Therefore, it has excellent characteristics in terms of productivity of biomass, pigments and lipid compared to other microalgae. The HS2 microalgae of the genus Chlorella were used that were deposited with the Korea Research Institute of Bioscience and Biotechnology under the accession number KCTC 13108BP.

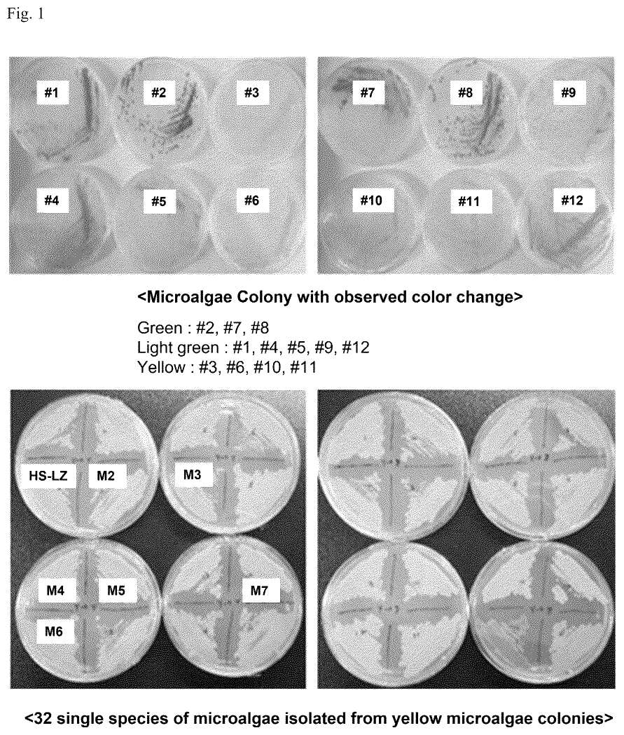

The HS2 microalgae of the genus Chlorella were cultured under dark condition, and then continuously sub-cultured for 3 days to obtain cells with lowest cell age. 10⁷ HS2 microalgal cells were inoculated in 10 ml of culture medium, treated with 1% (w/v) ethyl methanesulfonate (EMS, Sigma-Aldrich, Cat. No. M0880) at a concentration of 40 mM, and then the medium was cultured for 2 hours to induce mutation. Then, 10³ cells were smeared on each of BD Difco™ Yeast mold Agar solid medium (271210, BD Difco, USA). Candidate mutant microalgae colonies were selected by screening colonies in which color change occurred among a total of 50 solid media and streaking again (FIG. 1). The existing HS2 microalgae medium showed dark green, and the medium in which the color was changed to light green or yellow due to the changes in the composition of the pigment produced was selected.

The mutant microalgae selected through the color change in the solid medium were cultured again in a liquid medium supplemented with glucose as a carbon source, and the color change of the medium was additionally observed by setting both light and dark conditions, respectively.

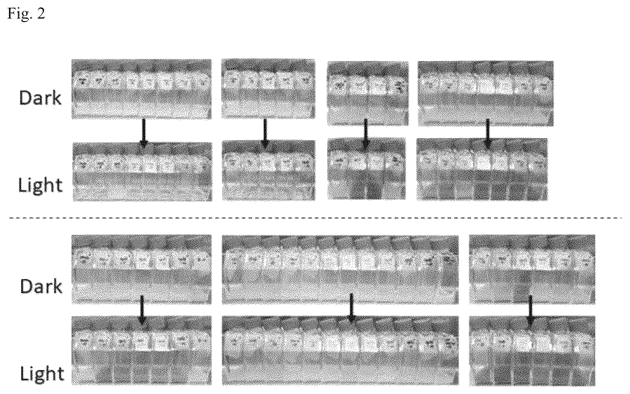

As a result, 7 mutants were selected in which the color of the liquid medium changed to dark yellow under both light conditions and dark conditions (FIG. 2), thereby it was possible to prepare and obtain the HS-LZ microalgae of the genus Chlorella of the present invention and other variants M2 to M7.

### Embodiment 2 Confirmation of Pigment Components of the HS-LZ Microalgae of the Genus Chlorella

Targeting the HS-LZ microalgae of the genus Chlorella of the present invention and other mutants M2 to M7, and the wild-type HS2 microalgae of the genus Chlorella prepared through Embodiment 1, by analyzing the components and content of the pigments they produce, the zeaxanthin and lutein productivity of the HS-LZ microalgae of the present invention was confirmed.

Specifically, 2 ml of culture medium of the HS-LZ microalgae, M2 to M7 mutants and wild-type HS 2 microalgae were centrifuged at 4° C. at 10,000 rpm per minute for 3 minutes and the supernatant was removed. And only the precipitated cells were lyophilized. Pigment extraction was carried out by adding 100 ml of ethanol to 1 g of lyophilized cells. In addition, components and contents of pigments produced from each microalgae were analyzed through high-performance liquid chromatography (HPLC, Agilent 1260 Infinity Binary LC system) (Column: Waters Spherisorb® S5 ODS1 4.6×250 mm, 51.1 μm Cartridge Column, Column temperature: 40° C., Injection amount: 20μl, Flow rate: 1.2 ml/min, Mobile phase A: acetonitrile 84%, methanol 2%, 0.1 M Tris-HCl 14%, Mobile phase B: methanol 68%, ethyl acetate 32%, Detector: Diode Array Detector).

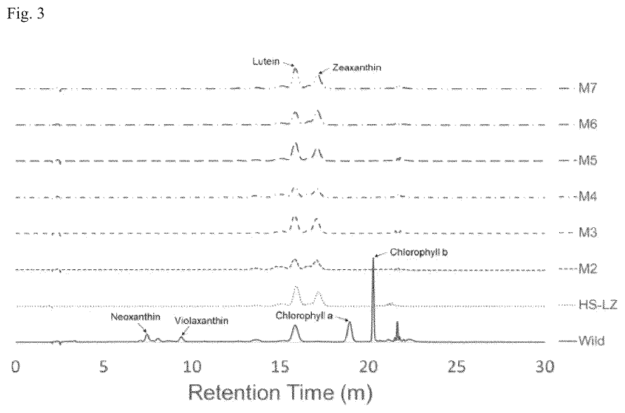

As a result, it was confirmed that the wild-type HS2 microalgae of the genus Chlorella contained lutein but did not contain zeaxanthin as a pigment component. In addition to lutein, other pigment components such as neoxanthin, violaxanthin, chlorophyll a and chlorophyll b were measured to be produced in large quantities. In contrast, in the case of the variants M2 to M7 and the HS-LZ microalgae of the genus Chlorella of the present invention selected through Embodiment 1, only peaks of lutein and zeaxanthin were appeared as a result of HPLC analysis. Accordingly, it was confirmed that other pigment types were hardly produced, with lutein and zeaxanthin accounting for the majority of the pigment contents (FIG. 3).

### Embodiment 3 Confirmation of zeaxanthin and lutein Productivity of the HS-LZ Microalgae in the Genus Chlorella

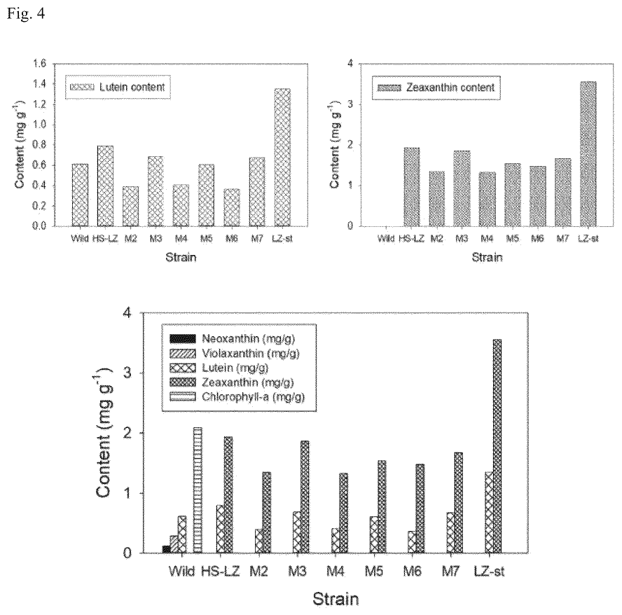

The zeaxanthin and lutein productivity of the HS-LZ microalgae of the genus Chlorella was confirmed. After drying the culture medium of the HS-LZ microalgae of the present invention, the variants M2 to M7 and wild-type HS2 microalgae of the genus Chlorella, the amounts of pigments contained per dry weight of the cell culture medium was calculated and compared based on the content of each pigment analyzed through Embodiment 2. In addition, the components and contents of the pigment were measured under stress conditions by additionally culturing the HS-LZ microalgae of the present invention for 7 days in a state in which glucose, an organic carbon source, was exhausted. And likewise, the amount of pigment per dry weight of the cell culture medium was calculated and compared (FIG. 4).

As a result, it was confirmed that the HS-LZ microalgae and the M2 to M7 variants of the present invention produced only zeaxanthin and lutein as pigment components and contained almost no other pigments. In particular, it was confirmed that the production amount of zeaxanthin was excellent. Specifically, it was measured that the HS-LZ microalgae of the present invention could produce 1.94 mg/g of zeaxanthin and 0.79 mg/g of lutein per dry weight of the culture medium. In contrast, in the case of wild-type HS2 microalgae, it was confirmed that lutein was partially produced, but zeaxanthin was not produced, and chlorophyll a was produced the most. Through the above experimental results, it was confirmed that the HS-LZ microalgae of the genus Chlorella of the present invention, unlike the existing HS2 microalgae of the genus Chlorella, have the ability to produce zeaxanthin. And it was confirmed that the productivity of zeaxanthin is excellent since a large amount of the pigment produced is zeaxanthin. In addition, by confirming that the HS-LZ microalgae could produce lutein with zeaxanthin but did not produce other pigments such as chlorophyll a, neoxanthin, and violaxanthin, the advantages of the present invention that the process of separating and purifying zeaxanthin and lutein separately is not necessary or can be simplified was confirmed.

Furthermore, it was confirmed that that the pigment productivity of the HS-LZ microalgae of the present invention is remarkably improved when it was cultured under stress conditions such as when the organic carbon source was consumed. Through this, it was confirmed that the HS-LZ of the genus Chlorella of the present invention could produce up to 3.56 mg/g of zeaxanthin and up to 1.35 mg/g of lutein per dry weight of culture medium, and thus, correspond to the microalgae having very good productivity for these pigments, zeaxanthin and lutein.

Although the present invention is described in detail only with respect to the described embodiments, it is obvious to those skilled in the art that various changes and modifications are possible within the scope of the technical spirit of the present invention, and that these changes and modifications fall within the scope of the appended claims.

### ACCESSION NUMBER

Depository Institution: Korea Research Institute of Bioscience and Biotechnology Biological Resources Center

Accession number: KCTC14308BP

Date: 20200911

1. HS-LZ microalgae of the genus Chlorella (Chlorella sp. HS-LZ) deposited under the accession number KCTC 14308BP.

2. The HS-LZ microalgae of claim 1, wherein the HS-LZ microalgae of the genus Chlorella are capable of producing zeaxanthin.

3. The HS-LZ microalgae of claim 1, wherein the pigments produced by the HS-LZ microalgae of the genus Chlorella contain at least one of zeaxanthin and lutein in an amount of 90 parts by weight or more based on 100 parts by weight of the pigments.

4. The HS-LZ microalgae of claim 1, wherein the pigments produced by the HS-LZ microalgae of the genus Chlorella does not contain at least one selected from the group consisting of chlorophyll a, chlorophyll b, neoxanthin and violaxanthin.

5. A method for producing zeaxanthin or lutein, comprising the steps of:
culturing the HS-LZ microalgae of the genus Chlorella (Chlorella sp. HS-LZ) deposited under the accession number KCTC 14308BP; and
obtaining pigments from the culture medium of the microalgae.

6. The method according to claim 5, wherein the step of culturing the microalgae comprises culturing the microalgae for 3 to 14 days under conditions in the absence of an organic carbon source.

7. The method according to claim 5, wherein the step of obtaining the pigments comprises the step of disrupting microalgal cells in the culture medium or the step of extracting the pigments from the microalgae.

8. The method according to claim 7, wherein the method does not comprise the step of removing chlorophyll a, chlorophyll b, neoxanthin and violaxanthin.

9. A composition for producing zeaxanthin or lutein comprising the HS-LZ microalgae of claim 1.

10. A pharmaceutical composition for preventing or treating eye diseases, comprising the HS-LZ microalgae of claim 1 or an extract thereof.

11. A health functional food for preventing or improving eye diseases, comprising the HS-LZ microalgae of claim 1 or an extract thereof.


SEQUENCE LISTING (1 sequence)

SEQ ID NO: 1 - DNA, 1606 bp, Artificial Sequence
  HS-LZ 18s rDNA
ctgctttatactgtgaaactgcgaatggctcattaaatcagttatagtttatttgatggtacctactactcggatacccgtagtaaatctagagctaatacgtgcgtaaatcccgacttctggaagggacgtatttattagataaaaggccgaccgggctctgcccgactcgcggtgaatcatgataacttcacgaatcgcatggccttgtgccggcgatgtttcattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgacacagggaggtagtgacaataaataacaatactgggccttttcaggtctggtaattggaatgagtacaatctaaaccccttaacgaggatcaattggagggcaagtctggtgccagcagccgcggtaattccagctccaatagcgtatatttaagttgctgcagttaaaaagctcgtagttggatttcgggtggggcctgccggtccgccgtttcggtgtgcactggcagggcccaccttgttgccggggacgggctcctgggcttcactgtccgggactcggagtcggcgctgttactttgagtaaattagagtgttcaaagcaggcctacgctctgaatacattagcatggaataacacgataggactctggcctatcctgttggtctgtaggaccggagtaatgattaagagggacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcctagtctcaaccataaacgatgccgactagggatcggcggatgtttcttcgatgactccgccggcaccttatgagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacatagtgaggattgacagattgagagctctttcttgattctatgggtggtggtgcatggccgttcttagttggtgggttgccttgtcaggttgattccggtaacgaacgagacctcagcctgctaaatagtcacggttggttctccagccggcggacttcttagagggactattggcgactagccaatggaagcatgaggcaataacaggtctgtgatgcccttagatgttctgggccgcacgcgcgctacactgatgcattcaacgagcctagccttggccgagaggcccgggtaatctttgaaactgcatcgtgatggggatagattattgcaattattaatcttcaacgaggaatgcctagtaagcgcaagtcatcagcttgcgttgattacgtccctgccctttgtacacaccgcccgtcgctcctaccgattgggtgtgctggtgaagtg